(19) United States
(12) Patent Application Publication
(10) Pub. No.: US 2007/0231878 A1
(43) Pub. Date: Oct. 4, 2007

(54) Collagen of fish scale and method of making thereof

(75) Inventors: Chwen-Herng Wu, Keelung City (TW); Huey-Jine Chai, Keelung City (TW)

Correspondence Address: PAI PATENT & TRADEMARK LAW FIRM, 1001 FOURTH AVENUE, SUITE 3200, SEATTLE, WA, 98154, US

(73) Assignee: Fisheries Research Institute

(21) Appl. No.: 11/255,087

(22) Filed: Oct. 20, 2005

(30) Foreign Application Priority Data
Oct. 27, 2004 (TW) 093132627

(51) Int. Cl. C07K 1/00

(52) U.S. Cl. 435/273

(57) ABSTRACT

This invention discloses a method for making collagen of fish scale, and more specifically it relates to a method of using enzyme to extract collagen of fish scale, the method comprising: washing and heating the fish scale material; smashing the fish scale material; adding the protein hydrolase into the fish scale material, and then affecting it in warm water to become hydrolyte; centrifugation the hydrolyte; taking out the supernatant of the hydrolyte rice; and drying the supernatant to become collagen powder. Said collagen of fish scale made from said method is a high produce rate, high purity and low pollution collagen of fish scale.

### BACKGROUND OF THE INVENTION

1. Field of the Invention

This invention relates generally to the collagen of fish scale and the method of making the same, and more specifically it relates to a method of using enzyme to extract collagen of fish scale.

2. Description of the Related Art

The present invention is directed to a method of making collagen of fish scale.

The collagen made for cosmetics, food, medical article, industry, and etc. in the past is using from the extract of collagen tissue of cattle, pig, chicken, mammal and poultry animal. In recent years, the epidemic diseases of livestock and poultry occur continuously, like Bovine Spongiform Encephalopathy, Foot-and-mouth disease, and bird virus, so the safety of the collagen from livestock and poultry are faced challenge, and some users having allergic reaction when they use the collagen from livestock. So that, it has some safety problems when use the collagen from land animal.

As a result of said question, the higher safety fish collagen gains more attention in recent years, in which the fish skin and fish scale have more collagen quantity, and they use to be material for making collagen frequently. But, the collagen from fish skin has its special odor, high quantity fat and turbid matter, causes high opacity, so that the odor and turbid matter will be limited its using category when the collagen be the composition of cosmetics.

In contract, fish scale has 50% collagen and 50% hydroxyaptite (HAp). In the past, the first step for extract collagen of fish scale is taken out fat by organic solvent, such as acetone, the second step is taken out the hydroxyaptite by acidity solution, and then the third step is filtered to gain the rude collagen. In JPO patent publication number P2004-91418A discloses a method in which the steps to extract fish scale collagen is taking out hydroxyaptite by hydrochloric acid, decompounding after adding water and drying. However, this method takes long time, makes with high cost, and injures the environment greatly, so it is not unfavorable for a large amount making. Therefore, many inventors prove the method by using enzyme for decompounding fish scale collagen. In JPO patent publication number P2003-327599A discloses a method in which the steps to extract fish scale collagen is three phases enzyme affecting after washing, taking out fat by acidity solution, and expanding fish scale, the fish scale collagen made from this method is a colorless, odorless and low turbid matter collagen. But this method still soaks fish scale material in hydrochloric acid, and extracts for 3 days by enzyme to gain fish scale collagen. Thereof, this method takes long time, and injures the environment greatly.

Therefore, there is a need of providing a method for making higher produce rate, lower pollution, and time-saving fish scale collagen. The fish scale collagen with these characteristic can reduce the possibility of economic and environmental dispute.

### SUMMARY OF THE INVENTION

The present invention advantageously fills the aforementioned need by providing a method for making collagen of fish scale.

The purpose of the present invention is to provide a method for making collagen of fish scale. More especially, it provides a method for making collagen of fish scale by enzyme extraction.

Another purpose of the present invention is to provide the collagen of fish scale which is made by the above-mentioned method.

The method for making collagen of fish scale according to the present invention comprises the following steps:

(a) Wash the fish scale and then heat the fish scale;

(b) Smash the fish scale from step (a) by using a machine;

(c) Add 1% protein hydrolase into the fish scale from step (b), and then affect it in warm water to become hydrolyte;

(d) Centrifugation the hydrolyte from step (c);

(e) Take out the supernatant from step (d);

(f) Dry the supernatant from step (e) to become powder.

Another purpose of the present invention is to provide the collagen of fish scale which is made by the above-mentioned method, the characteristic of said collagen of fish scale is a higher produce, higher purity, higher permeation, and lower pollution collagen of fish scale.

In said method for making collagen of fish scale, the raw material of the fish scale is preferably, but not limited to, raw fish scale and fish scale with skin.

The device of heating said fish scale used in the present invention is selected from, but not limited to, the group consisting of water baths, double boilers, pasteurizing machines, and pressure cookers.

The device of smashing said fish scale is selected from a device having physical smash function to smash the fish scale. The device used in the present invention is selected from, but not limited to, the group consisting of homogenizer, ultrasonic machine, smash machine, and food processor.

The present invention provides a method for mass producing collagen of fish scale which having higher purity, higher retrieve, and higher permeation rate. The collagen of fish scale made from the present invention method can be used as, but not limited to, cosmetics, personal care products, nutriments, health foods, and regular foods, etc.

The present invention now will be described more fully hereinafter with reference to the accompanying drawings, which are intended to be read in conjunction with both this summary, the detailed description and any preferred and/or particular embodiments specifically discussed.

### BRIEF DESCRIPTION OF THE DRAWINGS

The present invention will become apparent upon reading of the following detailed description of the present invention in conjunction with the drawings, as follows:

FIG. 1 is a block diagram of a method for making collagen of fish scale according to the present invention;

FIG. 2 is a diagram of distribution map showing the molecular weight of said collagen of fish scale after affected Protease N for 2.5 hours.

### DETAILED DESCRIPTION OF THE INVENTION

The present invention is directed to a method of making collagen of fish scale, and to the collagen of fish scale made by such a method.

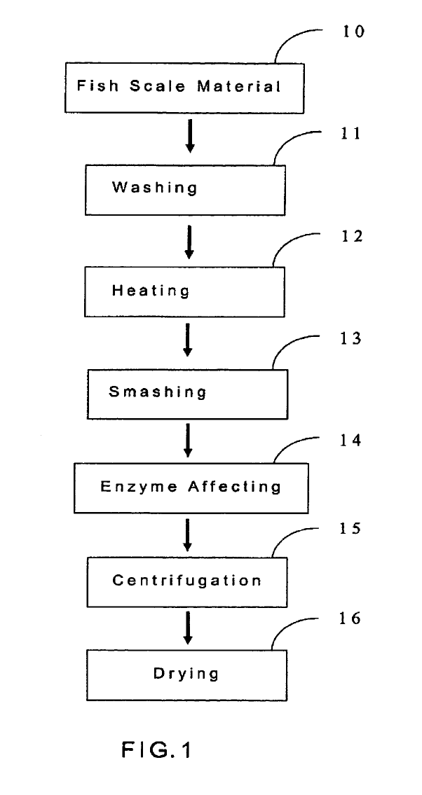

With reference to the FIG. 1, the steps of the method of the present invention are described in the following.

In Step a, fish scale material 10 is washed 11, then fish scale material 10 is heated 12. The fish scale material 10 of the present invention is selected from raw fish scale or fish scale with skin. The device for heating the fish scale of the present invention is selected from, but not limited to, the group consisting of water baths, double boilers, pasteurizing machines, and pressure cookers.

In Step b, the heated 12 fish scale material 10 is smashed 13 by a machine. The device used in the present invention is selected from, but not limited to, the group consisting of homogenizer, ultrasonic machine, smash machine, and food processor, in order to smash the fish scale.

In Step c, the smashed 13 fish scale material 10 from step b is added 1% protein hydrolase, and then it is affected 14 in warm water. The protein hydrolase for treating 14 fish scale material used in the present invention is selected from neutral enzyme.

In Step d, the enzyme affected 14 hydrolyle from step c is centrifugation 15.

In Step e, the centrifugation 15 hydrolyte from step d is taken out the supernatant of hydrolyte.

In Step f, the centrifugation 15 hydrolyte from step e is dried to become powder. The device used in the present invention is selected from, but not limited to, the group consisting of spray dryer, freezer, hot-air dryer, cold-air dryer, and decompression dryer. One specific embodiment of the present invention is dried by spray dryer.

The collagen of fish scale made from the present invention method can be used as, but not limited to, cosmetics, personal care products, nutriments, health foods, and regular foods, etc. However, its use is not limited to these applications.

### EXAMPLE 1

### Different Heating Time

1000 grams fish scale material is washed to rid of impurities. The material is divided into 5 group (each is 200 grams), and then each of 5 group is heated 0•15•30•45 and 60 minutes at 121° C., respectively. The heated fish scale materials are added 400 grams water to smash into small pieces by Disperser (Kinematica®), and then take out the supernatant by centrifugation (room temperature, 8,000 rpm, 30 minutes). The supernatant is dried by spray dryer to become powder type. The produce of collagen of fish scale of example 1 is showed in table 1.

TABLE 1
The produce of collagen of fish scale in different heating time

| TIME | Produce | Rude Collagen |
|---|---|---|
| (min) | (%) | (%) |
| 0 | 6 | 0.7 |
| 15 | 53 | 2.5 |
| 30 | 42 | 3.7 |
| 45 | 46 | 3.8 |
| 60 | 63 | 4.0 |

### EXAMPLE 2

### Different Heating Temperature

400 grams fish scale material is washed to rid of impurities. The material is divided into 2 group (each is 200 grams), and then each of 2 group is heated 15 minutes at 100° C. and 121° C., respectively. The heated fish scale materials are smashed into small pieces by Disperser (Kinematica®). Said materials are affected 2 hours at 50° C. after adding 400 grams water and 1% Protease N (PN, sigma), and then the hydrolyte is taken out the supernatant by centrifugation (room temperature, 8,000 rpm, 30 minutes). The supernatant is dried by spray dryer to become powder type. The produce and purity of collagen of fish scale of example 2 is showed in table 2.

TABLE 2
The produce and purity of collagen of fish scale in different heating
temperature

| Heating | Produce | Water | Rude Collagen | Purity |
|---|---|---|---|---|
| Temperature (□) | (%) | (%) | (%) | (%) |
| 100 | 85 | 92.9 | 6.3 | 88 |
| 121 | 94 | 91.9 | 7.2 | 89 |

### EXAMPLE 3

### Different Hydrolyzing Time

800 grams fish scale material is washed to rid of impurities. The material is divided into 4 group (each is 200 grams), and then 4 groups are heated 15 minutes at 121° C., respectively. The heated fish scale materials are smashed into small pieces by Disperser (Kinematica®). Each of said groups is affected 2 hours at 35•50˜60 and 75° C. after adding 400 grams water and 1% Protease N (PN, sigma), and then the hydrolyte is taken out the supernatant by centrifugation (room temperature, 8,000 rpm, 30 minutes). The supernatant is dried by spray dryer to become powder type. The produce and purity of collagen of fish scale of example 3 is showed in table 3.

TABLE 3
The produce and purity of collagen of fish scale in different
hydrolyzing temperature

| Temperature | Produce | Water | Rude Collagen | Purity |
|---|---|---|---|---|
| (□) | (%) | (%) | (%) | (%) |
| 35 | 85 | 92.4 | 6.4 | 85 |
| 50 | 93 | 92.4 | 6.6 | 87 |
| 60 | 62 | 93.1 | 5.9 | 85 |
| 75 | 78 | 88.0 | 10.7 | 89 |

### EXAMPLE 4

### Different Enzyme and Different Hydrolyzing Temperature

1,200 grams fish scale material is washed to rid of impurities. The material is divided into 6 group (each is 200 grams), and then 6 groups are heated 15 minutes at 121° C., respectively. The heated fish scale materials are smashed into small pieces by Disperser (Kinematica®). Each of said groups is affected 0.5•1.0•1.5•2.0•2.5 and 3.0 hours at 50° C. after adding 400 grams water and 1% Protease N (PN, sigma), and then the hydrolyte is taken out the supernatant by centrifugation (room temperature, 8,000 rpm, 30 minutes). The supernatant is dried by spray dryer to become powder type.

1,200 grams fish scale material is washed to rid of impurities. The material is divided into 6 group (each is 200 grams), and then 6 groups are heated 15 minutes at 121° C., respectively. The heated fish scale materials are smashed into small pieces by Disperser (Kinematica®). Each of said groups is affected 0.5•1.0•1.5•2.0•2.5 and 3.0 hours at 50° C. after adding 400 grams water and 1% Protamex (Bacillus, sigma), and then the hydrolyte is taken out the supernatant by centrifugation (room temperature, 8,000 rpm, 30 minutes). The supernatant is dried by spray dryer to become powder type.

1,200 grams fish scale material is washed to rid of impurities. The material is divided into 6 group (each is 200 grams), and then 6 groups are heated 15 minutes at 121° C., respectively. The heated fish scale materials are smashed into small pieces by Disperser (Kinematica®). Each of said groups is affected 0.5•1.0•1.5•2.0•2.5 and 3.0 hours at 50° C. after adding 400 grams water and 1% Flavourzyme (Aspergillus oryzae, sigma), and then the hydrolyte is taken out the supernatant by centrifugation (room temperature, 8,000 rpm, 30 minutes). The supernatant is dried by spray dryer to become powder type.

200 grams fish scale material is washed to rid of impurities. The material is heated 15 minutes at 121° C. The heated fish scale material is smashed into small pieces by Disperser (Kinematica®). Said material is affected 2.0 hours at 50° C. after adding 400 grams water and 1% Protease N (PN, sigma), and then it is affected 0.5 hours with 1% Flavourzyme (Aspergillus oryzae, sigma). The hydrolyte is taken out the supernatant by centrifugation (room temperature, 8,000 rpm, 30 minutes). The supernatant is dried by spray dryer to become powder type.

The produce of collagen of fish scale of example 4 is showed in table 4, and the purity of collagen of fish scale of example 4 is showed in table 5.

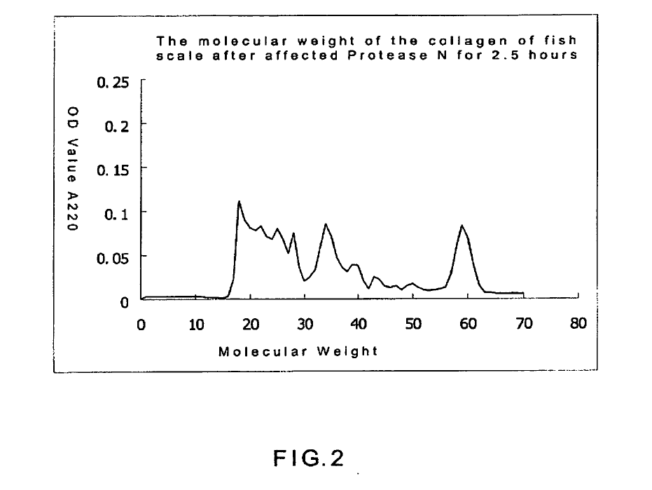

The molecular weight of the collagen of fish scale after affected Protease N for 2.5 hours is showed in FIG. 2 and table 7.

TABLE 4
The produce of collagen of fish scale in different enzyme and
different hydrolyzing temperature

| | Time | Produce |
|---|---|---|
| Enzyme | (hours) | (%) |
| Protease N | 0.5 | 79 |
| | 1 | 86 |
| | 1.5 | 88 |
| | 2 | 93 |
| | 2.5 | 95 |
| | 3 | 93 |
| Protamex | 0.5 | 56 |
| | 1 | 56 |
| | 1.5 | 58 |
| | 2 | 53 |
| | 2.5 | 62 |
| | 3 | 58 |
| Flavourzyme | 0.5 | 69 |
| | 1 | 82 |
| | 1.5 | 81 |
| | 2 | 83 |
| | 2.5 | 71 |
| | 3 | 78 |
| Protease N | 2.5 | 93 |
| 2 hours + Flavourzyme | | |
| 0.5 hours | | |

TABLE 5
The purity of collagen of fish scale in different enzyme and different
hydrolyzing temperature

| | Time | Water | Rude | Purity |
|---|---|---|---|---|
| enzyme | (hours) | (%) | collagen(%) | (%) |
| Protease N | 0.5 | 92.9 | 6.6 | 92 |
| | 1 | 93.1 | 6.3 | 91 |
| | 1.5 | 92.8 | 6.6 | 93 |
| | 2 | 92.2 | 7.2 | 93 |
| | 2.5 | 91.9 | 7.8 | 97 |
| | 3 | 90.8 | 8.5 | 93 |
| Protamex | 0.5 | 94.5 | 5.0 | 90 |
| | 1 | 94.4 | 4.9 | 88 |
| | 1.5 | 93.5 | 5.7 | 88 |
| | 2 | 94.1 | 5.1 | 86 |
| | 2.5 | 92.9 | 6.3 | 88 |
| | 3 | 93.3 | 5.9 | 88 |
| Flavourzyme | 0.5 | 93.2 | 6.3 | 92 |
| | 1 | 93.5 | 6.0 | 92 |
| | 1.5 | 92.8 | 6.5 | 91 |
| | 2 | 92.6 | 6.7 | 91 |
| | 2.5 | 93.7 | 5.7 | 90 |
| | 3 | 92.6 | 6.7 | 90 |
| Protease N | 2.5 | 92.7 | 6.6 | 91 |
| 2 hours + Flavourzyme | | | | |
| 0.5 hours | | | | |

### COMPARATIVE EXAMPLE

A. Using 0.5 M Acetic Acid for Extract Collagen Overnight

200 grams fish scale material is washed to rid of impurities. The material is extracted overnight by 0.5M acetic acid. The extracted fish scale material is taken out the supernatant by centrifugation (room temperature, 8,000 rpm, 30 minutes). The supernatant is dried by spray dryer to become powder type.

B. Fish Scale be Hydrolyzed without Heating and Smashing

200 grams fish scale material is washed to rid of impurities. The material is affected 2.0 hours at 50° C. after adding 400 grams water and 1% Protease N (PN, sigma), and then the hydrolyte is taken out the supernatant by centrifugation (room temperature, 8,000 rpm, 30 minutes). The supernatant is dried by spray dryer to become powder type.

C. Fish Scale be Hydrolyzed and Smashed without Heating

200 grams fish scale material is washed to rid of impurities. The material is smashed into small pieces by Disperser (Kinematica®). Said material is affected 2.0 hours at 50° C. after adding 400 grams water and 1% Protease N (PN, sigma), and then the hydrolyte is taken out the supernatant by centrifugation (room temperature, 8,000 rpm, 30 minutes). The supernatant is dried by spray dryer to become powder type.

The produce and purity of collagen of fish scale of comparative example is showed in table 6.

TABLE 6
The produce and purity of collagen of fish scale of comparative
example

| | Produce | Water | Rude | Purity |
|---|---|---|---|---|
| Example | (%) | (%) | Collagen (%) | (%) |
| A | 3 | 99.2 | 0.03 | 4 |
| B | 39 | 95.7 | 2.6 | 59 |
| C | 54 | 95.1 | 4.5 | 73 |

TABLE 7
The molecular weight of the collagen of fish scale after affected
Protease N for 2.5 hours

| Molecular | | | | |
|---|---|---|---|---|
| Weight | | | | Protein Quantity |
| Range | | | OD Value | (%) |
| | >5 | KD | 0.54 | 24.5 |
| | 2-5 | KD | 0.399 | 18.1 |
| | 1-2 | KD | 0.522 | 23.7 |
| | <1 | KD | 0.392 | 17.8 |

| | |
|---|---|
| <5 KD (<2 KD) | 59.5 |

| | | |
|---|---|---|
| | | (41.0) |
| TOTAL | 1.853 | 84% |

The experiment result shows that the produce rate of the collagen of fish scale heating at 121° C. is higher than the produce rate heating at 100° C. (the produce rate is 94% and 85%, respectively); the produce rate of the collagen of fish scale by Protease N hydrolyzing 2-3 hours is 93-97%. In contrast, the produce rate of the collagen of fish scale by 0.5 M acetic acid extracting overnight is 3%, the produce rate of the collagen of fish scale by hydrolyzing without heating and smashing is 39%, and the produce rate of the collagen of fish scale by hydrolyzing and smashing without heating is 54% in comparative example. It is important, the produce rate of the collagen of fish scale made from the method of the present invention is more than 90%, and the produce rate of the collagen of fish scale is significantly higher than the produce rate of the collagen of fish scale made from the method of comparative example.

The purity of the collagen of fish scale heated in different heating temperature is 88% and 89%, respectively. The purity of the collagen of fish scale affected in different hydrolyzing temperature is 85-89%. The purity of the collagen of fish scale affected in 1% Flavourzyme for 0.5 to 3 hours is 90-92%; the purity of the collagen of fish scale affected in 1% Protease N for 0.5 to 3 hours is 91-97%, in which the purity of the collagen of fish scale affected in 1% Protease N for 2.5 hours is the highest. In contrasts the purity of the collagen of fish scale by 0.5 M acetic acid extracting overnight is 4%, the purity of the collagen of fish scale by hydrolyzing without heating and smashing is 59%, and the purity of the collagen of fish scale by hydrolyzing and smashing without heating is 73% in comparative example. It is important, the purity of the collagen of fish scale made from the method of the present invention is more than 90%, and the purity of the collagen of fish scale is significantly higher than the purity of the collagen of fish scale made from the method of comparative example.

FIG. 2 and table 7 are disclosed the molecular weight of the collagen of fish scale after affected Protease N for 2.5 hours. It is important, the molecular weight less than 2 KD of the collagen of fish scale made from the method of the present invention is 41.0%; the molecular weight less than 5 KD of the collagen of fish scale is 59.5%. Namely, the collagens of fish scale made from the method of the present invention have small molecular weight, and relatively, this collagen of fish scale have good permeability for skin.

The method of the present invention for making collagen of fish scale spends less than 6 hours from washing fish scale material, heating, smashing, enzyme hydrolyzing to centrifugation, Compared with prior art, the method of present invention cut down the produce time (3 days is cut down to become 6 hours). Notably, the method disclosed in the present invention can produce more collagen of fish scale in the same time.

While the present invention has been described above in terms of specific embodiments, it is to be understood that the invention is not limited to these disclosed embodiments. Many modifications and other embodiments of the invention will come to mind of those skilled in the art to which this invention pertains; they are intended to be and are covered by both this disclosure and the appended claims. It is intended that the scope of the invention should be determined by proper interpretation and construction of the appended claims and their legal equivalents, as understood by those skilled in the art relying upon the disclosure in this specification and the attached drawings

What is claimed is:

1. A method for making collagen of fish scale, comprising the steps of:
(a) washing the fish scale material, then heating the fish scale material;
(b) smashing said fish scale material from step (a) by smash machine;
(c) adding 1% protein hydrolase into said fish scale from step (b), and then affecting it in warm water to become hydrolyte;
(d) centrifugation said hydrolyte from step (c);
(e) taking out the supernatant of said hydrolyte from step (d); and
(f) drying said supernatant from step (e) to become collagen powder.

2. The method of claim 1, wherein said fish scale material is a raw fish scale.

3. The method of claim 1, wherein said fish scale material is a fish scale with skin.

4. The method of claim 1, wherein the heating temperature in step (a) is 100-121° C.

5. The method of claim 1, wherein said smash machine in step (b) is using a disperser to smash the said fish scale material.

6. The method of claim 1, wherein said protein hydrolase in step (c) is one protein hydrolase.

7. The method of claim 1, wherein said protein hydrolase in step (c) is mixing two kinds of protein hydrolase.

8. The method of claim 6, wherein said protein hydrolase is a neutral protein hydrolase.

9. The method of claim 7, wherein said protein hydrolase is a neutral protein hydrolase.

10. The method of claim 1, wherein said drying step in step (f) is using spray dryer.

11. A collagen of fish scale made from said method of claim 1, wherein said collagen of fish scale is a small molecular weight collagen of fish scale which the produce rate and purity are more than 90%.
